(19) United States
(12) Patent Application Publication
(10) Pub. No.: US 2004/0022753 A1
(43) Pub. Date: Feb. 5, 2004

(54) External application for enhancing the skin permeability of the active components therein

(76) Inventors: Eui Seok Shin, Kyungki-do (KR); Jang Young Lee, Seoul (KR); Mu Sung Kim, Kyungki-do (KR); Sung Gu Lee, Pladal-ku (KR); Dong Chul Lee, Kyungki-do (KR); Byung Young Kang, Seoul (KR)

Correspondence Address: Powell Goldstein Frazer & Murphy, P O Box 97223, Washington, DC, 20090-7223, US

(21) Appl. No.: 10/276,176

(22) PCT Filed: May 16, 2001
(86) PCT No.: PCT/KR01/00788

(30) Foreign Application Priority Data
May 16, 2000 (KR) 2000/26086

(51) Int. Cl. A61K 38/48; A61K 31/715; A61K 7/06; A61K 7/11

(52) U.S. Cl. 424/70.13; 424/94.63; 514/54; 424/70.14

(57) ABSTRACT

The present invention relates to a composition for external application enhancing the skin absorption of the active components infused in the composition, more specifically relates to an external application having enhanced skin permeability of the active components by using protease stabilized by β-1,3-glucan branched with β-1,6-linkage as an agent for enhancing the skin permeability to weaken the wall properties of the corneous layer.

### FIELD OF THE INVENTION

The present invention relates to an external application for enhancing the skin absorbency of the active agent infused therein, more specifically, relates to an external application having enhanced skin absorbency of the active agents by using protease stabilized by β-1,3-glucan branched with β-1,6-linkage as an agent for enhancing the skin absorption.

### BACKGROUND OF THE INVENTION

Skin is divided into three portions, a stratum corneum, epidermal layer and dermis, and the most important barrier for skin absorption of the active agent contained in the external application such as cosmetics is the stratum corneum located on the outermost portion.

Especially, because the stratum corneum is composed of corneous cell mainly comprising keratins and lipid layer filling the space between corneous cells, liposoluble active agents are well absorbed into the skin while water-soluble agents and large-molecule agents are not. Therefore, most of the agents infused in the external applications such as cosmetics are not easily absorbed into the skin, because they are water-soluble.

Conventionally, to solve these problems, using non-polar solvents, surfactants, and lipid acid, et al. as skin absorption enhancers is disclosed. [Chen L H, Chien Y W, “Enhancement of skin penetration” In “Novel cosmetic delivery systems” 1990, 60; Rhein L D, Robbins C R, Fernee K, Cantore R “Surfactant structure effects on swelling of isolated human stratum corneum”, J. Soc. Cosmet Chem. 1986, 37: 125; Cooper E R, Merritt E W, Smith R L., “Effect of fatty acids and alcohols on the penetration of acylclovir across human skin in vitro,” J. Pharm. Sci. 1985; 74: 688]. However, although these skin absorption enhancers enhance the skin-absorption of the active agent, they cause fierce skin stimuli after being absorbed into the skin because their molecular weights are small.

Further, applying sonophoresis or minute iontophoresis on the skin to enhance the skin absorption of the water-soluble materials to the skin is also disclosed. [Sloan J B, Slotani K. “Iontophoresis in dermatology: a review” J. Am. Acad. Dermatol. 1986, 4:671; Langer R. “Ultrasound-mediated transdermal protein delivery” Science 1995; 269:850] However, because these methods need expensive equipments, these methods are not practically used in the external applications such as cosmetic compositions.

In addition, some patents teach other methods for enhancing the skin absorption of the active agents bad modifying or weakening the skin barrier function with enzyme. For example, U.S. Pat. Nos. 5,534,260 and 5,296,222 show methods for enhancing the absorption of the active agents by adding protease. However, the above methods are just adding protease into the composition, and therefore, the protease is degenerated or loses its activity with time by the effects of other elements infused in the formulation. As can be estimated above, because the enzyme (active agent) does not accomplish enhanced skin absorption, the above methods are not suitable for external application comprising a lot of elements in addition to the active agent.

To solve above-mentioned problems, inventors of the present invention searched for a method for enhancing the skin absorption of the active agents infused in the external application such as cosmetics, and finally found that when protease stabilized by β-1,3-glucan branched with β-1,6-linkage is used as an agent for enhancing the skin absorption in the external application, the absorption rate is increased.

### SUMMARY OF THE INVENTION

Therefore, an object of the present invention is to provide a composition for external application to the skin having the properties of enhanced skin absorbency of the active agents infused in the composition.

### DETAILED DESCRIPTION OF THE MENTION

Hereinafter, the present invention is described in detail.

The present invention provides a composition for external skin application with enhanced skin absorption properties by using protease stabilized by β-1,3-glucan branched with β-1,6-linkage as skin absorption enhancing agent in the skin external application. Said composition enhances the skin absorption by modifying the structure of the corneous layer or removing the layer without causing skin stimulus.

A method for preparing the protease used as a skin absorption enhancing agent of the present composition complies with the method described in KR 2000-60771 A1(published on Oct. 16, 2000) entitled “Method for preparing stabilized enzyme or protein, and composition for external application comprising the stabilized enzyme or protein provided by the method”. In detail, the method for stabilizing the protease comprises the steps of; (1) reacting a glucan with periodates to transform the β-1,6-linkage of the glucan into aldehyde; (2) removing unreacted periodates from the reaction solution of step (1); (3) adding a protease to the reaction solution of step (2) in an amount of 0.00001˜10.0% by weight; (4) adding a reducing agent to the reaction solution of step (3) in an amount of 0.0001˜1.0% by weight; and (5) washing the product of step (4).

Glucans used in the above method are β-1,3-glucans branched with β-1,6-linkage, and the glucans can make functional groups only in the β-1,6-linkage depending on the chemical reaction. Glucans used in the present invention are not restricted, preferably schizophyllan derived from Schizopyllum commune, scleroglucan derived from Sclerotinia sp. and lentinan derived from Lentinus edodes are used.

In addition, proteases stabilized by the method of the present invention are not restricted, preferably papain, chymotrypsin, trypsin, carboxypepidase, pepsin, et al. are used.

The amount of the stabilized protease contained in the composition of the external application is 0.0001˜99.9999% by weight on the basis of the weight of total composition. Further, formulation for external application is not restricted. For example, they may have cosmetic formulation such as skin softener, nutrition water, massage cream, make-up base, lipstick, pack, gel, shampoo, rinse, hair tonic or soap, or external dermatological formulation such as lotion, ointment, gel, cream, patch or spray.

In addition, the sorts of active agents having their skin absorption properties enhanced by the skin absorption enhancers of the present invention are conventional active agents, and are not restricted specifically. For example, water-soluble derivatives of L-ascorbic acid such as L-ascorbic acid, L-ascorbic phosphate, L-ascorbic glucoside, et al., vitamin B, kojic acid, kojic caffeine acid, kojic aminopropyl phosphate, hydroquinone, albutine, water soluble natural extract derived from green tea or grape by water or ethanol, et al. are used as active agents.

Hereinafter, the present invention is described more specifically with the embodiments of the present invention, however, the scope of the present invention is not restricted within the embodiments.

### DESCRIPTION OF THE PREFERRED EMBODIMENTS

### REFERENCE EXAMPLE 1

### Preparation of Stabilized Papain

1) To 50 ml of 0.5wt % aqueous solution of sschizophyllan (molecular weight of 2,000,000), 2 g of sodium iodide peroxide (NaIO₄) was added, and the resulting mixture was kept in the dark at 4° C. for 1 hour with stirring.

2) The resulting schizophyllan solution was subjected to dialysis over membrane (MW cut off 10,000) in the dark for 48 hours. The dialysis was carried out with replacements of 2500 ml water every 12 hours.

3) When the volume of the schizophyllan solution was increased to about 100 ml, 0.1wt % of papain was added, and the resulting solution was stirred for 2 hours in the dark at 4° C.

4) 0.05 g of sodium borohydride (NaBH₄) was added to the schizophyllan −papain solution, and followed by stirring for 4 hours. Then, 0.1 g of lysine was added and the resulting mixture was stirred for 2 hours in the dark at 4° C.

5) The resulting schizophyllan-papain solution was subjected to the dialysis as described in the step 2) to obtain a solution of papain coupled to schizophyllan (GE-1). The yield of the GE-1 prepared was 95% based on the enzyme activity.

EXAMPLE 1 AND COMPARATIVE EXAMPLE 1 AND 2
Water-gel type cosmestics

| | | | Comparative | Comparative |
|---|---|---|---|---|
| Ingredients | | Example 1 | Examle 1 | Example 2 |
| 1. | glycerin | 7.0 | 7.0 | 7.0 |
| 2. | Carboxyvinyl Polymer | 0.5 | 0.5 | 0.5 |
| 3. | Butylene Glycol | 3.0 | 3.0 | 3.0 |
| 4. | EDTA-Na₂ | 0.2 | 0.2 | 0.2 |
| 5. | Stabilized Papain | 1.0 | — | — |
| | (Reference Example 1, | | | |
| | GE-1) | | | |
| 6. | Papain | — | — | 1.0 |
| 7. | Kojic acid | 0.5 | 0.5 | 0.5 |
| 8 | Calcium hydroxide | 0.05 | 0.05 | 0.05 |
| 9. | Ethanol | 5.0 | 5.0 | 5.0 |
| 10. | Preservatives | q.s. | q.s. | q.s. |
| 11. | PEG-60 hardened caster oil | 0.3 | 0.3 | 0.3 |
| 12. | Perfumes | q.s. | q.s. | q.s. |
| 13. | Distilled water | To | To | To |
| | | 100 | 100 | 100 |

<Method for Preparation>

1) Ingredients 1˜7 were added to the distilled water, then completely dissolved by stirring.

2) Ingredients 10˜12 were added to the ethanol, then completely dissolved by stirring.

3) Solution prepared in step 2) was slowly added to the solution prepared in step 1) while stifling the solution.

4) Ingredient 8 was added to the solution of step 3) to form a gel.

5) Gas bubbles were removed by vacuum deaeration, then a water-gel type cosmestic was obtained.

EXAMPLE 2 AND COMPARATIVE EXAMPLE 3 AND 4
Water-in-Oil type emulsion cosmestics

| | | | Comparative | Comparative |
|---|---|---|---|---|
| Ingredients | | Example 2 | Example 3 | Example 4 |
| 1. | Stearic acid | 1.0 | 1.0 | 1.0 |
| 2. | Cetostearyl alcohol | 0.7 | 0.7 | 0.7 |
| 3. | Microcrystalline wax | 0.2 | 0.2 | 0.2 |
| 4. | Monostearic acid glycerin | 0.5 | 0.5 | 0.5 |
| 5. | Fluid paraffin | 5.0 | 5.0 | 5.0 |
| 6. | Monostearic acid sorbitan | 0.3 | 0.3 | 0.3 |
| 7. | Squalane | 3.5 | 3.5 | 3.5 |
| 8. | Distilled water | To | To | To |
| | | 100 | 100 | 100 |
| 9. | Dense glycerin | 6.5 | 6.5 | 6.5 |
| 10. | Hyaluronic acid extract | 0.5 | 0.5 | 0.5 |
| 11. | Sepigel 305 | 0.7 | 0.7 | 0.7 |
| 12. | Stabilized Papain | 1.0 | — | — |
| | (Reference Example 1, | | | |
| | GE-1) | | | |
| 13. | Papain | — | — | 1.0 |
| 14. | Ascorbic acid | 0.5 | 0.5 | 0.5 |
| 15. | Perfumes | q.s. | q.s. | q.s. |
| 16. | Preservatives | q.s. | q.s. | q.s. |

<Method for Preparation>

1) Component A (ingredient 1˜7) and Component B (ingredient 8˜10) were heated to 75° C.

2) A was slowly added to B while stirring at 7,000 rpm. After completely adding A to B, ingredients 14 and 16 were added to the mixture immediately, then stirred for two minute to emulsify.

3) Ingredients 11 and 15 were added and stirred for two minute to emulsify.

4) Mixture of 3) was stirred for 1 minute at 2,500 rpm to remove air.

5) Mixture of 4) was cooled in an ice bath to 28˜30° C.

6) Ingredient, 12 or 13 was added to the above cooled mixture, then stirred with 2,000 rpm.

7) The product was left to stand at room temperature for 24 hours to obtain a stabilized water-in-oil type cosmetic composition.

### EXPERIMENTAL EXAMPLE 1

### Measurement for Skin Absorption of the Kojic Acid

Measurement for skin absorption of the kojic acid was practiced in the Frantz permeation cells using the skins of a guinea pig. Abdominal skin was obtained from the guinea pig before the experiment. Above obtained skin was cut to a dimension of 1 cm², and stood in the permeation cell having the diameter of the permeation lens 0.9 cm, then held by the clamp. 0.5 ml of cosmetic compositions from Example 1 and Comparative Example 1 to be tested were applied on one side of the skin, while the other side of the skin was contacted with solvent mixture of distilled water and glycerin with the ratio of 1:1. The temperature was maintained at 32° C., which is the skin temperature. The solvent was collected periodically (with fixed time interval; hour), then the amount of the kojic acid absorbed into the skin was measured by HPLC. The results are shown in Table 1.

TABLE 1
Skin absorption of the kojic acid per applying density(μm/cm²/wt %)

| Time(hour) | Example 1 | Comparative Example 1 |
|---|---|---|
| 0 | 0 | 0 |
| 4 | 6.15 | 1.42 |
| 8 | 12.98 | 3.09 |
| 24 | 36.13 | 9.11 |

Considering that the duration time of general make-up is 4˜8 hours, table 1 shows that when a stabilized protease is added to the composition, the skin absorption of kojic acid is increased by about four times compared with a case that a stabilized protease is not added.

### EXPERIMENTAL EXAMPLE 2

### >Measurement for Skin Absorption of the Ascorbic Acid

The amount of the ascorbic acid absorbed into the skin was measured with the method described in Experimental Example 1 except that water-in-oil type cosmetic composition of Example 2 and Comparative Example 3 were used. The results are shown in Table 2.

TABLE 2
Skin absorption-of the ascorbic acid(μm/cm²/wt %)

| Time(hour) | Example 2 | Comparative Example 3 |
|---|---|---|
| 0 | 0 | 0 |
| 4 | 3.67 | 0.67 |
| 8 | 7.05 | 1.29 |
| 24 | 20.93 | 4.47 |

Table 2 shows that when a stabilized protease is added to the composition, the skin absorption of ascorbic acid is increased by about six times compared with a case that a stabilized protease is not added.

EXAMPLE 3 AND COMPARATIVE EXAMPLE 5 AND 6
Ointment

| | | | Comparative | Comparative |
|---|---|---|---|---|
| Ingredient | | Example 3 | Example 5 | Example 6 |
| 1. | Bee wax | 10.0 | 10.0 | 10.0 |
| 2. | Polysorbate 60 | 5.0 | 5.0 | 5.0 |
| 3. | PGE-60 hardened castor oil | 2.0 | 2.0 | 2.0 |
| 4. | Sorbitan Cesquioleate | 0.5 | 0.5 | 0.5 |
| 5. | Vaseline | 5.0 | 5.0 | 5.0 |
| 6. | Fluid Paraffin | 10.0 | 10.0 | 10.0 |
| 7. | Squalane | 5.0 | 5.0 | 5.0 |
| 8. | Shear butter | 3.0 | 3.0 | 3.0 |
| 9. | Carprylic/Capric | 5.0 | 5.0 | 5.0 |
| | triglyceride | | | |
| 10. | Glycerin | 10.0 | 10.0 | 10.0 |
| 11. | Propylene glycol | 5.0 | 5.0 | 5.0 |
| 12. | Triethanolamine | 0.2 | 0.2 | 0.2 |
| 13. | Perfumes | s.q. | s.q. | s.q. |
| 14. | Stabilized Papain | 2.0 | — | — |
| | (Reference Example 1, | | | |
| | GE-1) | | | |
| 15. | Papain | — | — | 2.0 |
| 16. | Hydroquinone | 2.0 | 2.0 | 2.0 |
| 17. | Distilled water | To | To | To |
| | | 100 | 100 | 100 |

<Method for Preparation>

1) A(ingredients 1˜9 and ingredient 17) and B (ingredients 10˜12) were heated to 75° C.

2) A was slowly added to B, and the mixture was stirred at 7,500 rpm for 5 minutes to emulsify.

3) Ingredient 13 was added to the mixture, and stirred at 7,500 rpm for 5 minutes to emulsify.

4) Mixture of 3) was cooled in an ice bath to 25° C.

5) Ingredients 14˜16 were added, and stirred at 2,500 rpm for mixing.

6) The product was left to stand at room temperature for 24 hours to obtain ointment.

### EXPERIMENTAL EXAMPLE 3

### Measurement for Skin Absorption of Hydroquinone

The amount of the hydroquinone absorbed into the skin was measured with the method described in Experimental Example 1 except that ointments prepared in the Example 3 and Comparative Example 5 were used. The results are shown in Table 3.

TABLE 3
Skin absorption of the hydroquinone (μm/cm²/wt %)

| Time(hour) | Example 3 | Comparative Example 5 |
|---|---|---|
| 0 | 0 | 0 |
| 4 | 18.66 | 3.67 |
| 8 | 37.05 | 6.99 |
| 24 | 109.43 | 19.47 |

Table 3 shows that when a stabilized protease is added to the composition, the skin absorption of hydroquinone is increased by about six times compared with a case that a stabilized protease is not added.

### EXPERIMENTAL EXAMPLE 4

### Measurement for Skin Absorption of the Kojic Acid Infused in the Aqueous Gel Type Cosmetic Composition After 30 Days of its Preparation

The amount of the kojic acid absorbed into the skin was measured with the method described in Experimental Example 1 except that aqueous gel type cosmetic compositions of Example 1 and Comparative Example 2 after 30 days of their preparation were used. The results are shown in Table 4.

TABLE 4
Skin absorption of the kojic acid (μm/cm²/wt %)

| Time(hour) | Example 1 | Comparative Example 1 |
|---|---|---|
| 0 | 0 | 0 |
| 4 | 6.35 | 1.33 |
| 8 | 13.02 | 3.10 |
| 24 | 36.93 | 9.20 |

Table 4 shows that although papain is contained in the cosmetic composition, as shown in case of comparative example 2 that do not comprise stabilized papain, papain loses its activity after 30 days of its preparation by the effects of the other ingredients contained together, therefore papain no longer shows the skin absorption enhancement.

### EXPERIMENTAL EXAMPLE 5

### Measurement for Skin Absorption of the Ascorbic Acid Infused in the Water-in-oil Cosmetic Composition After 30 Days of its Preparation

The amount of the ascorbic acid absorbed into the skin was measured with the method described in Experimental Example 2 except that water-in-oil type cosmetic compositions of Example 2 and Comparative Example 4 after 30 days of their preparation were used. The results are shown in Table 5.

TABLE 5
Skin absorption of the ascorbic acid (μm/cm²/wt %)

| Time(hour) | Example 1 | Comparative Example 1 |
|---|---|---|
| 0 | 0 | 0 |
| 4 | 4.64 | 0.62 |
| 8 | 7.22 | 1.33 |
| 24 | 21.54 | 4.58 |

Table 5 shows that although papain is contained in the cosmetic composition, as shown in the case of comparative example 4 that does not comprise stabilized papain, papain loses its activity after 30 days of its preparation by the effects of the other ingredients contained together, therefore papain no longer shows the skin absorption enhancement.

### EXPERIMENTAL EXAMPLE 6

### Measurement for Skin Absorption of Hydroquinone Confused in the Ointment After 30 Days of its Preparation

The amount of the hydroquinone absorbed into the skin was measured with the method described in Experimental Example 3 except that ointments of Example 3 and Comparative Example 6 after 30 days of their preparation were used. The results are shown in Table 6.

TABLE 6
Skin absorption of the hydroquinone (μm/cm²/wt %)

| Time(hour) | Example 1 | Comparative Example 1 |
|---|---|---|
| 0 | 0 | 0 |
| 4 | 18.51 | 3.55 |
| 8 | 36.89 | 7.02 |
| 24 | 110.25 | 20.21 |

Table 6 shows that although papain is contained in the cosmetic composition, as shown in case of comparative example 6 that does not comprise stabilized papain, papain loses its activity after 30 days of its preparation by the effects of the other ingredients contained together, therefore papain no longer shows the skin absorption enhancement.

### EXPERIMENTAL EXAMPLE 7

### Observation of the Skin Injury

The external applications prepared in Examples 1 to 3 were applied on the skin of the guinea pig as described in the Experimental Example 1. When 24 hours had passed after applying, the skin was taken out from the Frantz permeation cell to be observed with naked eyes.

As the result of the observation, skin injury was not observed in the case of Examples 1 to 3, and it was found that stabilized protease do not cause skin injury but enhances skin absorption of the active agents.

### EXPERIMENTAL EXAMPLE 8

### Skin Stabilization

To examine the skin stabilization function of the protease, 300 ml of the external applications prepared in Examples 1 to 3 were applied on the same position of the back of nude mouse once a day for 30 days to observe the skin injuries such as swelling, erythema, et al. with naked eyes. However, no skin injury was observed.

The skin absorption enhancers of the present invention do not cause skin problems such as skin stimuli, skin injury, et al., but the do enhance the skin absorption of the components that have problems in permeation because of they have low affinity to the corneous layer or high molecular weight.

1. A composition for external application comprising at least one active agent, wherein the skin absorption of said active agent is enhanced by comprising a protease that is stabilized by a skin absorption enhancing agent β-1,3-glucan branched with β-1,6-linkage.

2. A composition of claim 1, wherein said active agent is selected from the group consisting of L-ascorbic acid, derivatives of L-ascorbic acid, vitamin B, kojic acid, kojic caffeine acid, kojic aminopropyl phosphate, hydroquinone, albutine and water soluble natural.

3. A composition of claim 1, wherein said β-1,3-glucan branched with β-1,6-linkage is schizophyllan, scleroglucan or lentinan.

4. A composition of claim 1, wherein said proteases is selected from the group consisting of papain, trypsin, chymotrypsin, carboxypepidase and pepsin.

5. A composition of claim 1, wherein said proteases is stabilized by the method comprising the steps of;
(1) reacting β-1,3-glucan branched with β-1,6-linkage and periodates to transform the β-1,6-linkage of the glucan into aldehyde;
(2) removing unreacted periodates from the reaction solution of step (1);
(3) adding a protease to the reaction solution of step (2) in an amount of 0.00001˜10.0% by weight;
(4) adding a reducing agent to the reaction solution of step (3) in an amount of 0.0001˜1.0% by weight; and
(5) washing the product of step (4).
